(19) United States
(12) Patent Application Publication
(10) Pub. No.: US 2026/0328688 A1
(43) Pub. Date: Oct. 22, 2026

(54) ENHANCED DETECTION OF POLY- AND PERFLUOROALKYL SUBSTANCES IN POTABLE WATER USING LUMINESCENT SODIUM ALGINATE HYDROGEL-CARBON DOTS SENSOR

(71) Applicant: Board of Regents, The University of Texas System, Austin, TX (US)

(72) Inventors: Somayeh Mohammadi, El Paso, TX (US); Hamidreza Sharifan, El Paso, TX (US)

(21) Appl. No.: 19/468,332

(22) Filed: Feb. 3, 2026

Related U.S. Application Data
(60) Provisional application No. 63/755,429, filed on Feb. 7, 2025.

(51) Int. Cl. G01N 21/64; C08L 5/04; G01N 33/18

(52) U.S. Cl. CPC G01N 21/643; C08L 5/04; G01N 33/18; G01N 2021/6439

(57) ABSTRACT

Sensitive and selective detection platform is based on a sodium alginate (SA) hydrogel modified with fluorinated carbon dots (F-CDs) to enhance the detection of PFOA. The SA hydrogel-F-CDs composite exhibited strong fluorescence at 480 nm under optimal conditions, achieving a detection limit as low as 0.001 ppt. The sensor was characterized using SEM, FT-IR, PXRD, XPS, and zeta potential, revealing its structural integrity, functional groups, and the surface charge of the SA hydrogel-F-CDs network. Sensitivity assessments demonstrated a linear fluorescence response to PFOA concentrations ranging from 1 to 66 ppq. Selectivity tests confirmed the sensor's ability to distinguish PFOA from other perfluorinated compounds, with minimal interference from other substances. This sensor offers a reliable, efficient, and highly sensitive platform for the detection of PFAS, demonstrating potential for real-world environmental monitoring applications.

### CROSS-REFERENCE TO RELATED APPLICATION

This application claims the benefit of U.S. Provisional Patent Application Ser. No. 63/755,429, filed Feb. 7, 2025, and entitled “Enhanced Detection of Poly- and Perfluoroalkyl Substances in Potable Water Using Luminescent Sodium Alginate Hydrogel-Carbon Dots Sensor,” which is incorporated herein by reference in its entirety for all purposes.

### STATEMENT AS TO RIGHTS TO INVENTIONS MADE UNDER FEDERALLY-SPONSORED RESEARCH OR DEVELOPMENT

This invention was made with government support under 2024-67022-42827 awarded by National Institute of Food and Agriculture within United States Department of Agriculture. The government has certain rights in the invention.

### BACKGROUND

Per- and polyfluoroalkyl substances (PFAS), including perfluorooctanoic acid (PFOA), are synthetic chemicals that have been widely used since the 1950s due to their remarkable chemical stability and resistance to degradation. With over 10,000 different formulations, PFAS possesses diverse physicochemical properties, such as variations in polarity, chain length, hydrophobicity, and surface charge. Their omniphobicity has made them invaluable in industries ranging from firefighting foams and nonstick cookware to food packaging and emulsifiers. However, these properties also make PFAS persistent in the environment, known as “forever chemicals” due to their accumulation in natural and engineered water systems. Well-established standard method for PFAS detection, such High-Performance Liquid Chromatography/Tandem Mass Spectrometry (HPLC-MS/MS), offer excellent sensitivity and accuracy, with detection limits as low as 0.53 to 2.8 ng/L for various PFAS molecules, and can even identify molecular structures at levels as low as 1 ag/μL. However, these methods have significant drawbacks, including high complexity, cost, and the need for specialized equipment and training. Additionally, samples must be sent to laboratories for analysis, making the process slow and impractical for widespread use. To overcome these challenges, new PFAS sensors based on diverse devices and nanomaterials are being developed, aiming to provide rapid, portable, low-cost, and user-friendly detection methods.

Hydrogels are three-dimensional networks of hydrophilic polymers that offer a promising approach for addressing contamination due to their high-water absorption capacity, tunable properties, and ability to encapsulate and remove contaminants such as heavy metals (e.g., arsenite, mercury, organic pollutants (e.g., dyes, pesticides), and emerging pollutants like per- and polyfluoroalkyl substances (PFAS) from water systems. These polymeric structures, capable of retaining large amounts of water, are often formed from both natural and synthetic polymers such as alginate, chitosan, cellulose, collagen, hyaluronic acid, gelatin, methacrylate gelatin, and PEG. Their functional groups, such as hydroxyl, amino, and carboxyl, exhibit strong interactions with nanomaterials and PFAS molecules, facilitating adsorption and detection, making them highly suitable for environmental applications in PFAS remediation and sensing. Alginate (ALG), a water-soluble polysaccharide composed of β-D-mannuronic acid (M) and α-L-guluronic acid (G), stands out among hydrogels for PFAS detection due to its natural abundance, biocompatibility, and ease of gelation through ionic crosslinking with calcium ions (Ca²⁺), which provides a stable matrix for adsorption. The carboxyl groups in alginate exhibit strong binding interactions with PFAS molecules, enhancing the hydrogel's selectivity and efficiency in capturing these contaminants. However, despite its effectiveness, alginate hydrogels have limitations in terms of mechanical strength and adsorption capacity.

To address these challenges, nanomaterials have been incorporated into alginate hydrogels, forming hydrogel nanocomposites with significantly improved properties. These nanocomposites enhance mechanical strength, increase adsorption efficiency, and provide additional functional sites for improved PFAS binding and detection. Carbon-based nanoparticles, such as carbon nanotubes (CNTs), graphene, and carbon dots (CDs), along with metallic and silica nanoparticles, are commonly used to reinforce hydrogel networks, improving their structural integrity, adsorption capabilities, and overall stability, making them more versatile and effective for environmental applications. CDs are zero-dimensional luminescent carbon nanomaterials recognized for their excellent fluorescence, low toxicity, cost-effective synthesis, and high sensitivity, making them promising materials for optical and electrochemical biosensors in detecting environmentally significant targets.

### SUMMARY

There is a need for the following embodiments of the present disclosure. Of course, the present disclosure is not limited to these embodiments.

Embodiments of the present disclosure align with and support the regulatory requirements set forth by the U.S. Environmental Protection Agency (EPA) regarding the presence of PFAS in drinking water. Specifically, the EPA has established health advisories and proposed regulations to limit PFAS (per- and polyfluoroalkyl substances) concentrations to levels that are protective of public health. The current interim health advisory levels for certain PFAS, such as perfluorooctanoic acid (PFOA), are set at extremely low thresholds down to parts per trillion (ppt).

The capability of embodiment of this disclosure to detect PFOA at concentrations as low as 0.001 ppt is well below the advisory limits, ensuring compliance with and exceeding the EPA's stringent water quality standards. This level of sensitivity enables proactive monitoring and supports efforts to maintain safe drinking water standards in line with emerging regulations. By facilitating the rapid and accurate detection of PFAS, this invention helps water management authorities and environmental agencies meet EPA requirements for protecting public health against the long-term effects of these persistent contaminants.

Embodiments of this disclosure can include a highly sensitive and selective device for detecting per- and polyfluoroalkyl substances (PFAS), particularly perfluorooctanoic acid (PFOA), in drinking water. Embodiments can include devices that utilize a sodium alginate (SA) hydrogel integrated with fluorinated carbon dots (F-CDs) that emit strong fluorescence when exposed to specific concentrations of PFOA. Embodiments can include sensors that achieve ultra-low detection limits, down to approximately 0.001 parts per trillion (ppt), and demonstrate a linear response over a range from approximately 1 to approximately 66 parts per quadrillion (ppq). Structural and functional assessments of the sensors confirm their reliability and efficiency, with minimal interference from other substances. The devices have proven applicability for real-world monitoring through spiked recovery tests in tap water, achieving high accuracy in detection. Embodiments can provide a rapid, efficient, and effective solution for monitoring PFAS contamination in environmental water sources, enhancing safety and public health.

Per- and polyfluoroalkyl substances (PFAS), including perfluorooctanoic acid (PFOA), are chemicals of growing concern due to their long-lasting presence in the environment, their resistance to breaking down, and their ability to accumulate in living organisms. Embodiments can include an innovative detection device that uses a modified gel, called a sodium alginate hydrogel with fluorinated carbon dots (F-CDs), to spot very low levels of PFOA in drinking water. This gel-based sensor lights up (fluoresces) when it detects PFOA, allowing for easy identification. The sensor can detect PFOA at extremely low levels, down to 0.001 parts per trillion (ppt), making it highly sensitive.

The device's structure and functionality were thoroughly tested with tools that examine its appearance, material composition, and electrical properties, ensuring that it works as intended. Tests showed that the device reliably detects PFOA concentrations between 1 and 66 parts per quadrillion (ppq), and it can specifically identify PFOA even when other similar substances are present. To prove its effectiveness, the sensor was used to test tap water samples from different areas that were spiked with known amounts of PFOA, resulting in successful detection rates of 94% to 106.6%. This technology provides an efficient and precise way to check for PFAS in drinking water, offering great potential for practical use in environmental monitoring.

According to an embodiment of the present disclosure, a method of making a sodium alginate hydrogel-carbon dot composite comprises synthesizing a plurality of fluorinated carbon dots via a hydrothermal route comprising reacting tetrafluoroterephthalic acid and polyethyleneimine; and adding the plurality of fluorinated carbon dots to a sodium alginate solution. According to another embodiment of the present disclosure, an apparatus for detection of poly- and perfluoroalkyl substances in water comprises a device comprising a sensor comprising a sodium alginate hydrogel-fluorinated carbon dots composite comprising fluorinated carbon dots. According to another embodiment of the present disclosure, a composition of matter comprises: a sodium alginate hydrogel-carbon dot composite comprising fluorinated carbon dots.

These, and other, embodiments of the present disclosure will be better appreciated and understood when considered in conjunction with the following description and the accompanying drawings. It should be understood, however, that the following description, while indicating various embodiments of the present disclosure and numerous specific details thereof, is given for the purpose of illustration and does not imply limitation. Many substitutions, modifications, additions and/or rearrangements may be made within the scope of embodiments of the present disclosure, and embodiments of the present disclosure include all such substitutions, modifications, additions and/or rearrangements.

### BRIEF DESCRIPTION OF THE DRAWINGS

The drawings accompanying and forming part of this specification are included to depict certain embodiments of the present disclosure. A clearer concept of the embodiments described in this application will be readily apparent by referring to the exemplary, and therefore nonlimiting, embodiments illustrated in the drawings (wherein identical reference numerals (if they occur in more than one view) designate the same elements).

FIGS. 1A-1F are SEM images of SA hydrogel (a, b, c) and SA hydrogel-F-CDs composite (d, e, f), dried at room temperature, shown at different magnifications (50 μm and 100 μm) in accordance with embodiments of the present disclosure.

FIGS. 2A-2H are SEM images of 3D SA hydrogel (a-d) and 3D SA hydrogel-F-CDs composite (e-h), freeze-dried, at different magnifications (500 μm and 1 mm) in accordance with embodiments of the present disclosure.

FIGS. 3A-3D are XPS analysis of the SA hydrogel. (a) High-resolution C1s spectrum; (b) High-resolution Ca 2p spectrum; (c) High-resolution O1s spectrum; (d) Survey spectrum of the SA hydrogel with peaks corresponding to O1s, C1s, and Ca2p, alongside the elemental composition in accordance with embodiments of the present disclosure.

FIGS. 4A-4F are high resolution XPS analysis of the SA hydrogel-F-CDs, (a) C1s, (b) Ca2p, (c) F1s, (d) N1s, (e) O1s, and (f) the full XPS spectrum with elemental composition in accordance with embodiments of the present disclosure.

FIGS. 5A-5B are (a) FT-IR spectra of alginate, hydrogel, and hydrogel-F-CDs; (b) PXRD patterns of SA hydrogel, and SA hydrogel-F-CDs composite in accordance with embodiments of the present disclosure.

FIGS. 6A-6B are UV-Vis absorbance spectra of the unmodified SA hydrogel (black) and the SA hydrogel modified with F-CDs (red) (a); Fluorescence excitation and emission spectra of SA hydrogel-F-CDs. The optimal excitation wavelength is observed at 360 nm, while the emission peaks at 480 nm (b) in accordance with embodiments of the present disclosure.

FIGS. 7A-7C are fluorescence emission spectra of the non-fluorescent SA hydrogel under varying excitation wavelengths (300-480 nm), showing negligible fluorescence intensity (a). Fluorescence emission spectra of the SA hydrogel embedded with F-CDs, exhibiting strong excitation-dependent fluorescence with a noticeable red shift in the emission peak as the excitation wavelength increases (b); Counter map of 0.25 g SA hydrogel F-CDs dispersed in deionized water (c) in accordance with embodiments of the present disclosure.

FIGS. 8A-8B are selectivity evaluation of the SA hydrogel-F-CD-based assay. Fluorescence intensity enhanced the ΔF_{F-F0} value of SA hydrogel-F-CDs in the presence of various perfluorinated compounds, including PFOS, PFPeA, PFHxA, PFHpA, PFNA, PFDA, HFUDA, a mixture of these compounds without PFOA, and PFOA (a). Enhanced fluorescence intensity (ΔF) of SA hydrogel-F-CDs in response to inorganic contaminants (b). The asterisk indicates the statistical differences in accordance with embodiments of the present disclosure.

FIGS. 9A-9B are calibration curve showing the relationship between the increase in fluorescence intensity (ΔF=F−F₀) and PFOA concentration in the range of 1 to 66 ppq (a). Fluorescence emission spectra of SA hydrogel F-CDs at different PFOA concentrations, demonstrating the increase in fluorescence intensity with increasing PFOA concentration (b) in accordance with embodiments of the present disclosure.

FIGS. 10A-10C are Zeta potential measurements of the synthesized hydrogels and their interaction with PFOA. Zeta potential of SA hydrogel, showing a value of −17.42±1.33 mV (a). Zeta potential of SA hydrogel-F-CDs, significantly reduced to −0.63=3.1 mV (b). Zeta potential of SA hydrogel-F-CDs in the presence of 0.1 ppb PFOA, indicating a shift to −13.91±1.05 mV, suggesting the interaction between PFOA and the hydrogel-F-CD matrix (c) in accordance with embodiments of the present disclosure.

FIG. 11 is a schematic of a method in accordance with embodiments of the present disclosure.

FIG. 12 is a schematic of an apparatus in accordance with embodiment of the present disclosure.

### DETAILED DESCRIPTION

Embodiments presented in the present disclosure and the various features and advantageous details thereof are explained more fully with reference to the nonlimiting embodiments that are illustrated in the accompanying drawings and detailed in the following description. Descriptions of well-known materials, techniques, components and equipment are omitted so as not to unnecessarily obscure the embodiments of the present disclosure in detail. It should be understood, however, that the detailed description and the specific examples are given by way of illustration only and not by way of limitation. Various substitutions, modifications, additions and/or rearrangements within the scope of the underlying inventive concept will become apparent to those skilled in the art from this disclosure.

Per- and polyfluoroalkyl substances (PFAS), especially perfluorooctanoic acid (PFOA), are emerging environmental and health concerns due to their persistence, resistance to degradation, and bioaccumulation. Embodiments can include a sensitive and selective detection platform based on a sodium alginate (SA) hydrogel modified with fluorinated carbon dots (F-CDs) to enhance the detection of PFOA. The SA hydrogel-F-CDs composite exhibited strong fluorescence at 480 nm under optimal conditions, achieving a detection limit as low as 0.001 ppt. The sensor was characterized using Scanning Electron Microscopy (SEM), Fourier-Transform Infrared Spectroscopy (FT-IR), Powder X-ray Diffraction (PXRD), X-ray Photoelectron Spectroscopy (XPS), and zeta potential, revealing its structural integrity, functional groups, and the surface charge of the SA hydrogel-F-CDs network. Sensitivity assessments demonstrated a linear fluorescence response to PFOA concentrations ranging from 1 to 66 ppq. Selectivity tests confirmed the sensor's ability to distinguish PFOA from other perfluorinated compounds, with minimal interference from other substances. The practical applicability of the sensor was validated using spiked recovery experiments with tap water samples from various locations, achieving recovery rates between 94% and 106.6%. This sensor offers a reliable, efficient, and highly sensitive platform for the detection of PFAS, demonstrating potential for real-world environmental monitoring applications.

Embodiments synthesized an SA hydrogel-F-CD to enhance detection sensitivity, reducing the limit of detection to 0.001 ppt. The resulting nanocomposite hydrogel was thoroughly characterized using SEM, FT-IR, PXRD, XPS, and zeta potential, confirming its structural integrity and functional properties. The enhanced hydrogel sensor was then successfully applied to detect PFOA levels in potable water samples collected from various locations across West Texas, demonstrating its practical utility for water quality monitoring.

Perfluorinated substances including perfluorooctanoic acid (PFOA) and perfluorooctanesulfonic acid (PFOS), along with sodium alginate (SA), and calcium chloride were purchased from Sigma-Aldrich (MO, USA). Additionally, perfluoropentanoic acid (PFPeA), perfluorohexanoic acid (PFHxA), perfluoroheptanoic acid (PFHpA), perfluorononanoic acid (PFNA), perfluorodecanoic acid (PFDA), and henicosafluoroundecanoic acid (HFUDA) were obtained from TCI (Portland, Oregon, USA).

TABLE 1
Characteristics of various PFAS compounds

| PFAS | Carbon Chain | Solubility | Molecular |
|---|---|---|---|
| Compound | Length | (mg/L) | Formula |
| PFOA | 8 | 9500 | C8HF15O2 |
| PFOS | 8 | 570 | C8HF17O3S |
| PFPeA | 5 | 4600 | C5HF9O2 |
| PFHxA | 6 | 3200 | C6HF11O2 |
| PFHpA | 7 | 2500 | C7HF13O2 |
| PFNA | 9 | 2100 | C9HF17O2 |
| PFDA | 10 | 1800 | C10HF19O2 |
| HFUDA | 11 | 1600 | C11HF21O2 |

### Preparation of SA Hydrogel

To prepare the hydrogel, 2 grams (g) of SA powder was dissolved in 100 mL of deionized water (DI) and stirred vigorously at 40° C. for three hours. The mixture was then filtered through a 0.2 μm filter. The resulting solution was added dropwise into 50 mL of 0.1 mol/L CaCl₂) solution while continuously stirring with a magnetic stirrer until it formed SA hydrogel. To ensure complete cross-linking, the hydrogel was soaked in the CaCl₂) solution for 6 hours in the dark at room temperature. Finally, the hydrogel was washed three times with DI water to remove excess CaCl₂) from the surface.

Encapsulation of F-CDs within SA Hydrogels

The main purpose of encapsulating F-CDs within the hydrogel matrix was to enhance their effectiveness and ensure sustained interaction for PFAS detection in aqueous environments. Briefly, to synthesize F-CDs encapsulated in SA hydrogel, 1 mL of F-CD (83.2 mg/mL) was added to 50 mL of filtered 2% (w/v) SA solution and stirred for 30 minutes. The mixture was then added dropwise into 50 mL of 0.1 mol/L CaCl₂) solution while continuously stirring with a magnetic stirrer, forming an F-CD-SA hydrogel. Like SA hydrogel synthesis, the hydrogel was placed in the CaCl₂) solution for 6 hours to ensure complete cross-linking, followed by washing three times with DI water. The synthesized F-CD SA hydrogel appears blue under 365 nm UV light.

### Surface Morphology and Microstructure Analysis of Hydrogel Beads

The surface morphology and microstructure of the synthesized 3D hydrogel beads was characterized using SEM (HITACHI SU 3500), followed by two different desiccation methods of air drying at room temperature and freeze-drying. The dried beads with average diameter range of (1.5-2 mm) were cut in half, mounted on carbon tape affixed to a cylindrical aluminum holder, and sputter-coated with a <1 nm layer of gold. The samples were then examined at four magnifications using an accelerating voltage of 15 kV. Fourier transform infrared (FT-IR) spectra were obtained using a Thermo Scientific Nicolet iS5 with the KBr pellet method for spectral acquisition. X-ray diffraction (XRD) analysis was performed over a 2θ range of 5° to 90° using a Bruker D8 Venture, equipped with dual-wavelength X-ray sources (high-intensity copper and molybdenum) and an advanced air-cooled photon II CPAD detector.

### Procedure for PFOA Detection

The assay for detecting PFOA was optimized by varying the mass of the F-CDs-SA hydrogel between 0.25 and 1.0 g. Different concentrations of PFOA were added to the solution, followed by a 60-minute incubation at 25° C. Fluorescence emission spectra (λₑₓ=370 nm) were then recorded for quantitative analysis, with excitation and emission slit widths set at 5 nm and a voltage of 350 V. The fluorescence response (ΔF) was calculated as the difference between the intensities of the F-CDs-SA hydrogel solution with (F) and without (F₀) PFOA. For the standard addition recovery tests, groundwater and tap water samples were boiled, cooled to room temperature, and filtered three times using a 0.22 μm membrane.

### Selectivity Test

To evaluate the selectivity of the fluorescent SA hydrogel with F-CDs, seven perfluorinated compounds-PFOS, PFPeA, PFHxA, PFHpA, PFNA, PFDA, HFUDA-along with a mixture of these compounds (excluding PFOA) were tested under optimal conditions as substitutes for PFOA. Additionally, to assess potential interference from predominant ionic compounds commonly found in water, NaNO₃, NaCl, KCl, CaCl₂), NaH₂PO₄, and Na₂HPO₄ were evaluated to investigate their effects on the selectivity of the hydrogel.

### Results and Discussion

To preserve the original properties, the cationic F-CDs were synthesized via a solvothermal reaction between tetrafluoroterephthalic acid (TFTA) and polyethyleneimine (PEI). In general, the ratio of TFTA to PEI can be from approximately 10:1 by weight to approximately 1:1 by weight (and all ratios therebetween), preferably approximately 3:1 by weight. The synthesized F-CDs were then added to a 2% SA solution, followed by the addition of calcium chloride as the crosslinking agent to form the final composite. This material retains the inherent properties of both the F-CDs and the hydrogel while demonstrating enhanced cooperative features. These improvements are due to the small particle size, the 3D confined hydrophilic channels, and the abundance of functional groups on the surface. F-CDs were integrated into the hydrogel, forming physical cross-links via hydrogen bonding between the amino groups of the F-CDs and the carboxyl groups of SA. The fluorescent F-CDs-SA hydrogels were freeze-dried for SEM, EDS, PXRD, and TGA analyses.

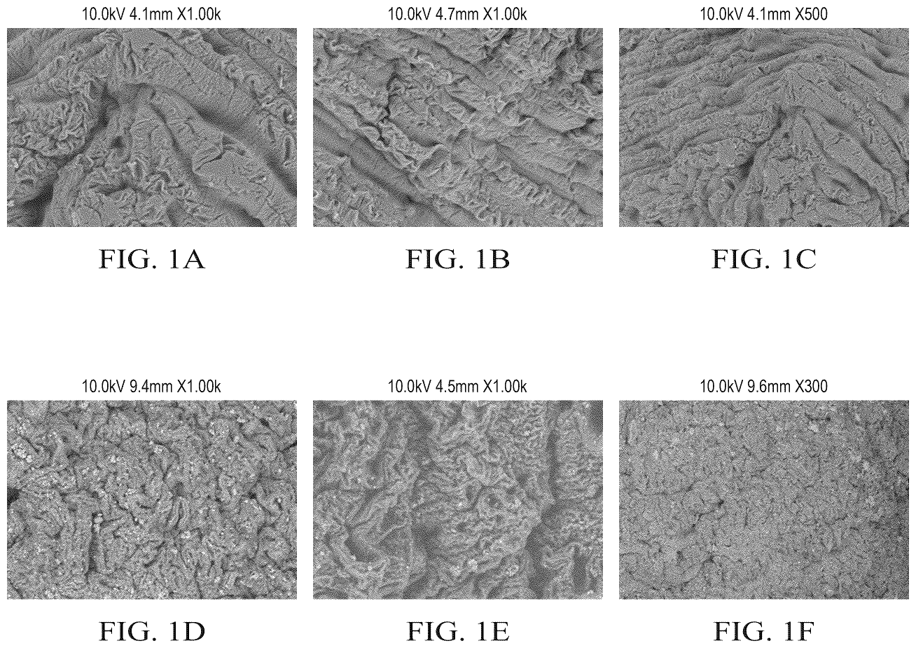

FIGS. 1A-1F illustrate the morphology of sodium alginate (SA) hydrogel and F-CD incorporated SA hydrogels as investigated under different drying regimes using SEM. The SEM analysis revealed drying method is a critical factor governing the structural integrity and porosity. In addition, impregnation with F-CDs at room temperature resulted in enhanced surface roughness and improved porosity, contributing to a more stable structure, which may facilitate higher adsorption efficiency for PFAS molecules.

The SA hydrogel dried in air at room temperature exhibited extensive expansion of surface area within compact folds in irregular converging rows. The microstructure was characterized by each row <70 μm and over 2 folds per 50 μm. However, the integration with synthesized F-CDs empowered the surface area of the SA physiochemically. The porosity was obviously improved with higher density of stacking folds per surface area and the F-CD functional groups allow them for enhanced interaction with PFAS molecules, facilitating better adsorption through electrostatic and fluorophilic interactions (FIGS. 1A-1C). This suggests that physical cross-links at nano scale between the F-CDs and the SA matrix stabilize the hydrogel network during the drying process (FIGS. 1D-1F).

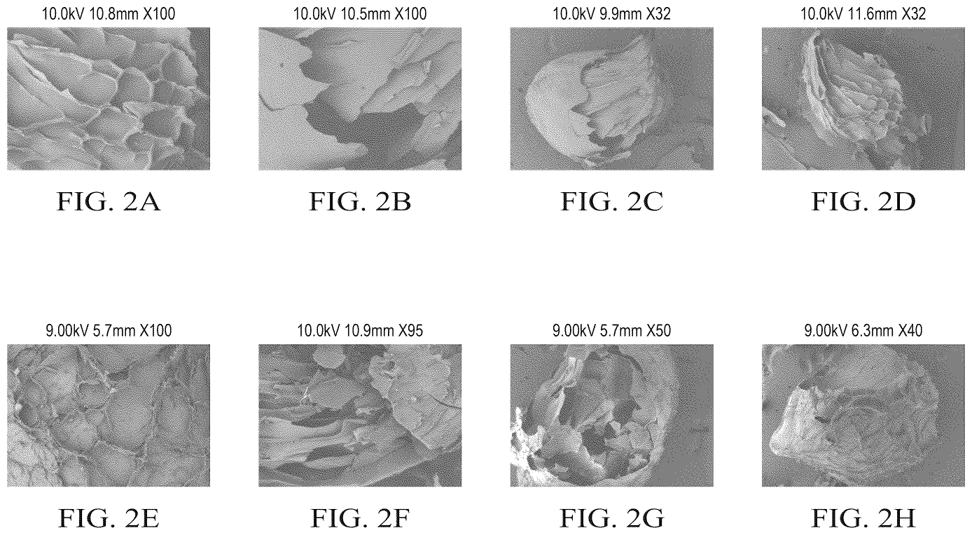

Unlike the air-drying method, the freeze-drying of SA hydrogel shown in FIGS. 2A-2D, resulted in a relatively more porous and less compressed network. The freeze-drying process, which removes water without deforming the structure, effectively preserved the internal architecture of the hydrogel, resulting in a well-defined pore structure. This method was more successful at maintaining the three-dimensional (3D) network of the hydrogel than air drying. The freeze-dried SA hydrogel-F-CDs, on the other hand, exhibited the most well-preserved and organized structure, with an extensive and highly porous network. The presence of F-CDs seemed to reinforce the hydrogel chemical reactivity, preserving its 3D architecture and preventing structural collapse during the freeze-drying process. The nano size and abundant functional groups of the F-CDs contribute to the enhanced stability and integrity of the hydrogel matrix (FIGS. 2E-2H).

The enhanced structural properties of the SA hydrogel-F-CDs, particularly in freeze-dried samples, make them promising candidates for detection and adsorption purposes which require high surface area and stability, such as sensing, drug delivery, or environmental remediation.

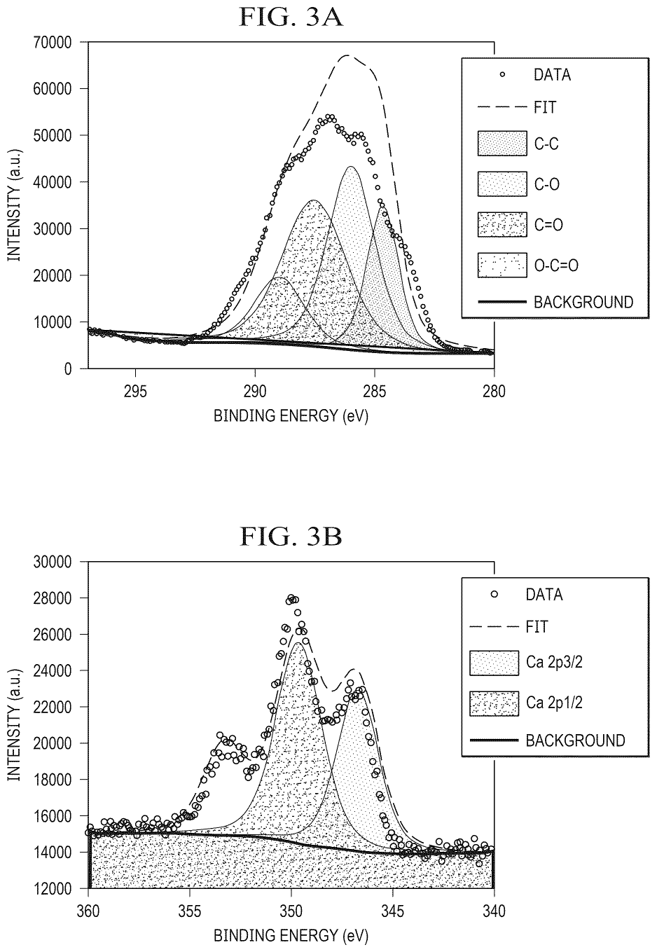

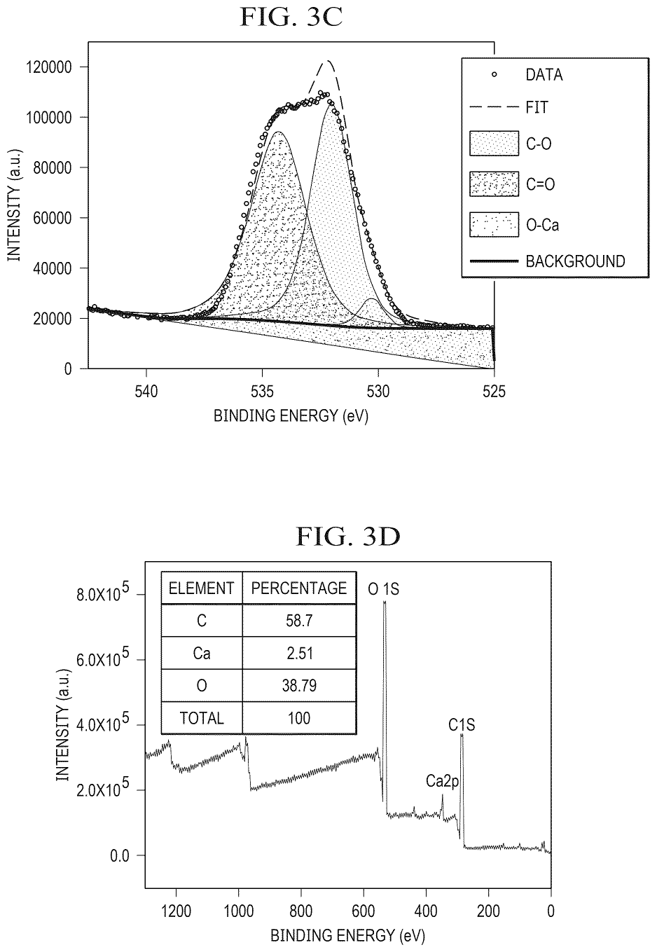

The XPS analysis of the SA hydrogel reveals carbon (C), oxygen (O), and calcium (Ca) as the key elements, with their respective chemical states identified through high-resolution scans. As shown in FIG. 3A, in the C1s spectrum, multiple peaks are observed, including C—C/C—H bonds (~285 eV), indicative of the hydrogel's organic backbone, C—O bonds (~286 eV) from hydroxyl or ether groups, and C═O and O—C═O bonds (~288-289 eV), which correspond to carbonyl and carboxylate groups typically present in the alginate structure. Furthermore, as illustrated in FIG. 3B, the Ca2p spectrum shows two prominent peaks at ~347 eV (Ca 2p3/2) and ~351 eV (Ca 2p1/2), confirming the presence of Ca²⁺ ions, which are responsible for the crosslinking in the hydrogel network. As depicted in FIG. 3C, the O1s spectrum further supports this, with peaks corresponding to C—O (~532 eV), C—O (~533 eV), and O—Ca (~531 eV) bonds, indicating the interaction between oxygen atoms and both carbon and calcium. The full XPS spectrum (FIG. 3D) shows that the prepared SA hydrogel consists of C, O, and Ca elements. As shown in the inset of FIG. 3D, the elemental composition reveals 58.7% C, 38.79% O, and 2.51% Ca, consistent with the expected structure of the SA hydrogel. This analysis highlights the crucial role of Ca²⁺ in crosslinking the alginate chains and confirms the expected chemical structure of the hydrogel.

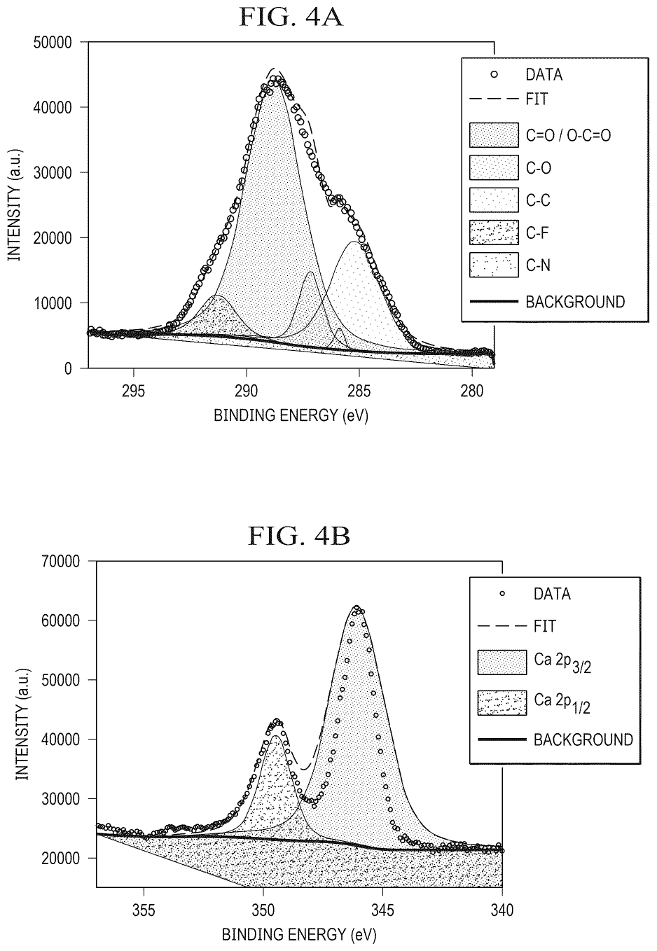

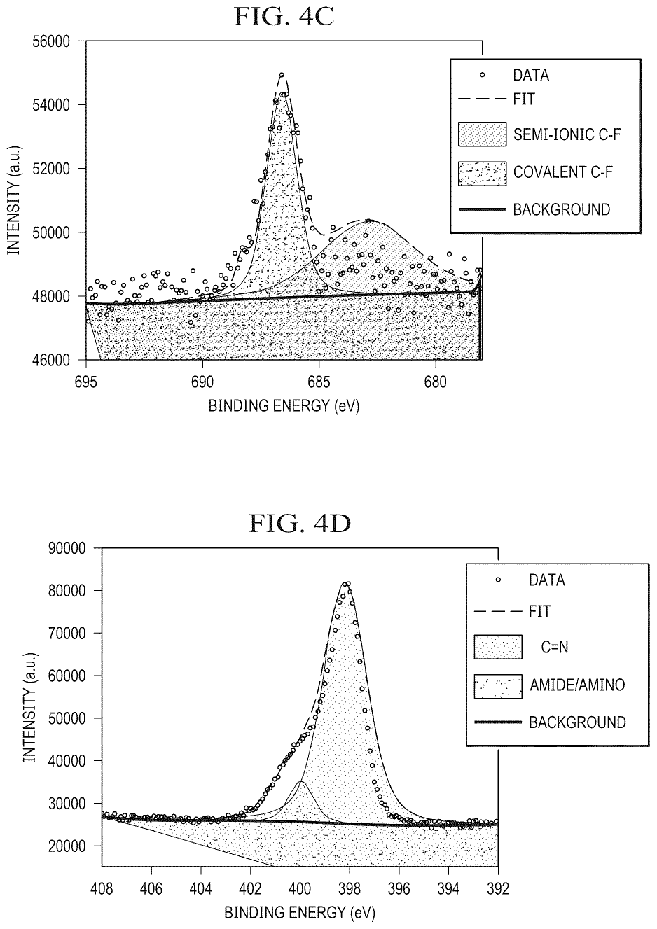

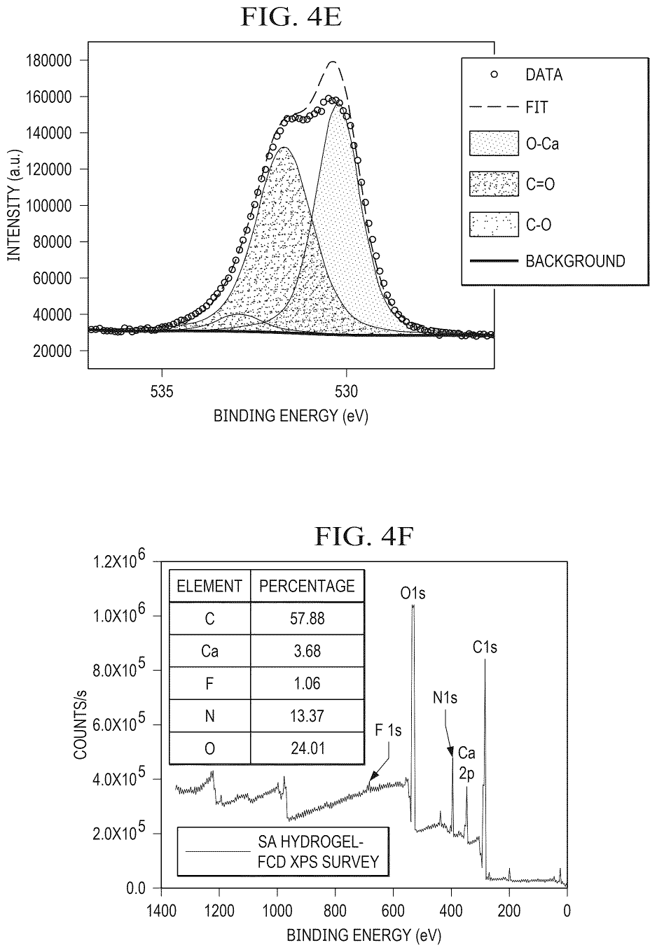

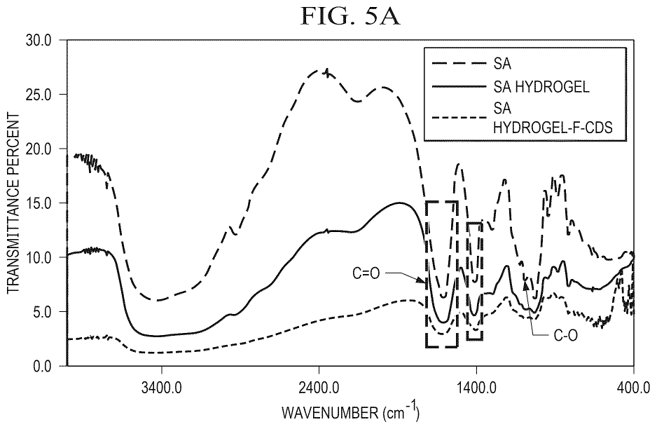

The XPS analysis of the SA hydrogel-F-CDs reveals the chemical states and elemental composition of key elements. The C1s high resolution spectrum (FIG. 4A) shows peaks for C═O/O—C═O (~288-289 eV), C—O (~286 eV), C—C(~285 eV), C—F, and C—N bonds. The Ca2p spectrum (FIG. 4B) confirms the presence of calcium ions, with peaks at ~347 eV (Ca 2p3/2) and ~351 eV (Ca 2p1/2), crucial for crosslinking within the hydrogel. The F1s spectrum (FIG. 4C) highlights semi-ionic and covalent C—F bonds, verifying the successful incorporation of fluorine, critical for PFAS detection. The N1s spectrum (FIG. 4D) reveals C═N and amide/amino groups, demonstrating the integration of nitrogen from the F-CDs. The O1s spectrum (FIG. 4E) shows peaks for O—Ca (~531 eV), C═O (~533 eV), and C—O (~532 eV), reflecting interactions between O, Ca, and C atoms that contribute to the hydrogel's structural integrity. Finally, the full XPS spectrum (FIG. 4F) reveals an elemental composition of 57.88% C, 24.01% O, 13.37% N, 3.68% Ca, and 1.06% F, confirming the successful incorporation of F-CDs into the SA hydrogel matrix, which enhances its functionality for PFAS detection through hydrogen bonding, fluorophilic and electrostatic interactions.

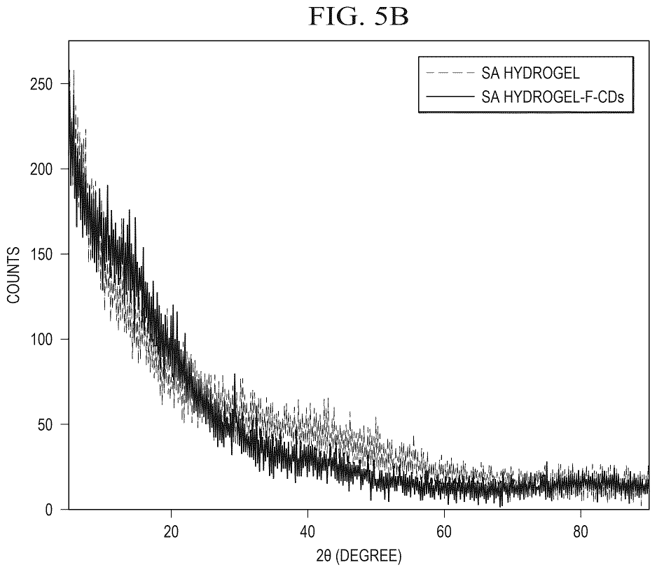

FTIR spectra of sodium alginate (SA), the SA hydrogel, and the SA hydrogel-F-CD composite are presented in FIG. 5A. In the spectrum of SA, the broad band around 3382.5 cm⁻¹ is attributed to O—H stretching vibrations, indicating the presence of hydroxyl groups. The peak at 1065.7 cm⁻¹ corresponds to C—O stretching vibrations. Additionally, the bands observed at 1621.4 cm⁻¹ and 1403.9 cm⁻¹ were assigned to the asymmetric and symmetric stretching vibrations of the carboxylate (C—O) groups, respectively. In the FTIR spectra of the SA hydrogel, the characteristic bands were shifted to 1640.88 cm⁻¹ and 1381.02 cm⁻¹, indicating cross-linking between the calcium ions and the carboxyl groups of SA. Similarly, in the FTIR spectra of the SA hydrogel-F-CD composite, these bands were further shifted to 1634.4 cm⁻¹ and 1397.4 cm⁻¹, respectively, reflecting the interaction between the hydrogel matrix and the F-CDs. FIG. 5B presents the PXRD patterns of F-CDS, SA hydrogel, and the SA hydrogel-F-CD composite. The F-CDs show a broad peak centered around 23°, characteristic of their amorphous structure due to its chemical amorphocility. The PXRD pattern of the SA hydrogel shows a broad peak, indicating its amorphous nature. Similarly, the SA hydrogel-F-CDs composite also exhibits a broad peak, suggesting the successful integration of F-CDs into the hydrogel matrix without affecting its amorphous structure. This confirms that both the hydrogel and the composite remain predominantly amorphous.

Hydrogen bonding facilitated the uniform dispersion of F-CDs throughout the gel network. Notably, the F-CDs served a dual function as they provided structural support within the hydrogel and acted as fluorophores, determining the overall emission properties of the material.

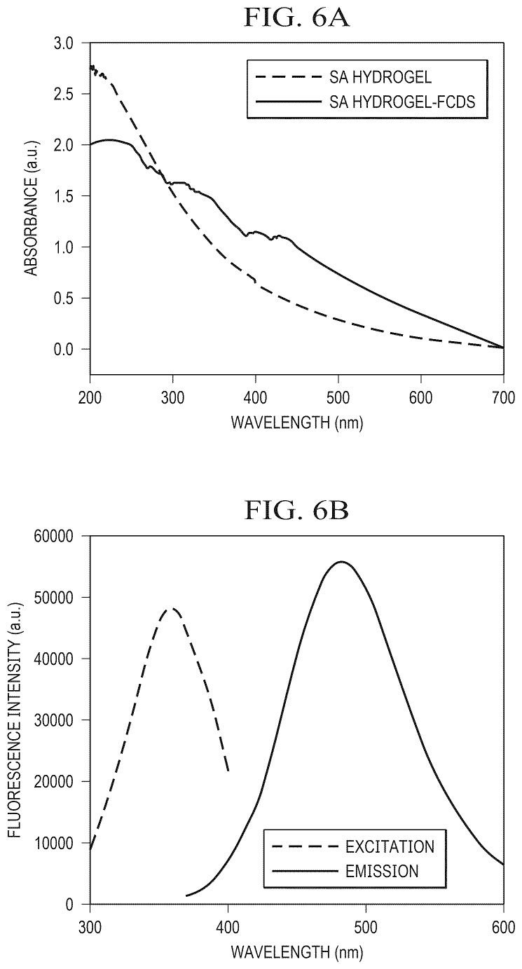

FIG. 6A shows the UV-Vis absorbance spectra of the unmodified SA hydrogel and the SA hydrogel-F-CDs. The SA hydrogel-F-CDs exhibits higher absorbance in the UV region (~250-400 nm) compared to the SA hydrogel, indicating the successful incorporation of F-CDs, consistent with SEM and FTIR results. This enhancement in the UV region confirms the modification of the SA hydrogel with F-CDs, improving its optical properties. The hydrogel-F-CDs exhibits optimal excitation at 360 nm, with a corresponding emission peak at 480 nm (FIG. 6B).

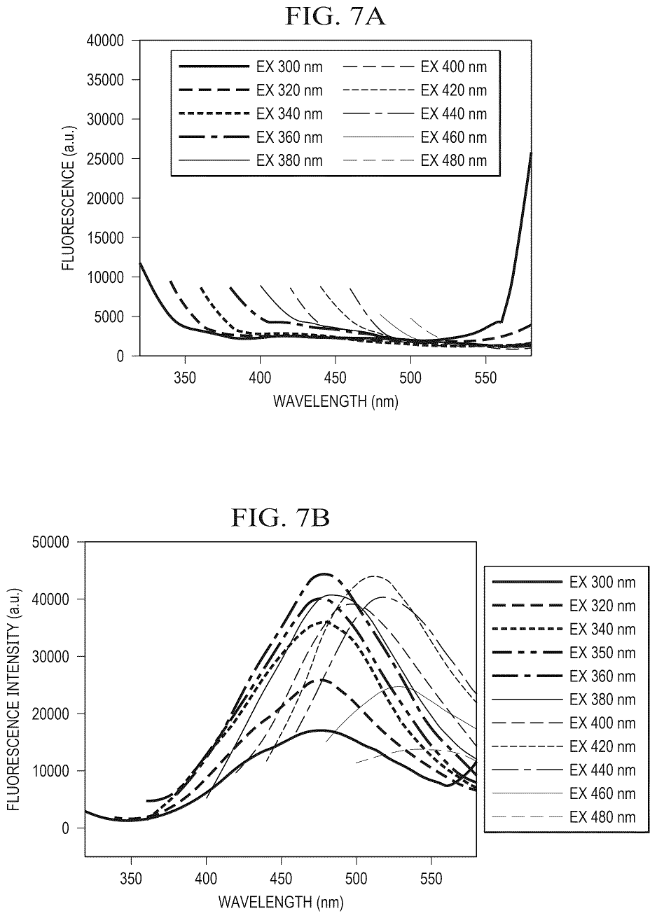

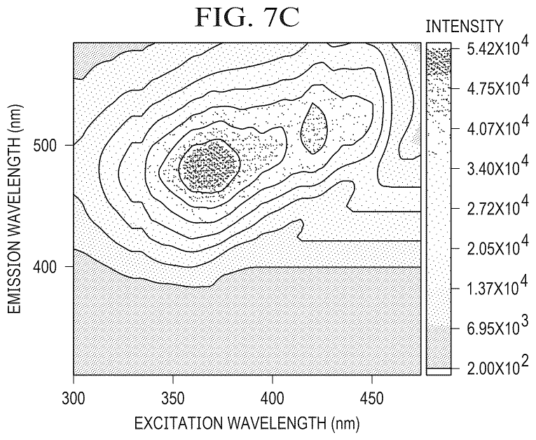

FIG. 7A represents the fluorescence emission spectra of a non-fluorescent SA hydrogel under varying excitation wavelengths. As observed, the hydrogel shows negligible fluorescence intensity across all excitation wavelengths (300-480 nm). This lack of fluorescence indicates that the SA hydrogel itself does not exhibit any inherent fluorescence properties. In contrast, FIG. 7B shows the fluorescence emission spectra of the SA hydrogel modified with F-CDs under different excitation wavelengths. Unlike the non-fluorescent SA hydrogel in FIG. 7A, the SA hydrogel-F-CDs exhibits strong fluorescence, and the emission peak is red-shifted with increasing excitation wavelengths, indicating its excitation-dependent fluorescence behavior. This suggests that the F-CDs are responsible for the optical properties of the hydrogel and that the material's fluorescence can be modulated by adjusting the excitation wavelength. This comparison clearly highlights the fluorescence enhancement resulting from the incorporation of F-CDs into the SA hydrogel matrix, making it highly suitable for detecting fluorine-containing substances, such as PFOA, through fluorophilic interactions. As shown in FIG. 7C, the 3D fluorescence contour plot presents the excitation-emission matrix of the SA hydrogel-F-CDs, where the optimal excitation wavelength is around 360 nm, leading to a maximum emission at approximately 480 nm. The highest intensity region (red) demonstrates the strong fluorescent properties of the composite.

### Selectivity Assessment of Fluorescent SA Hydrogel F-CDs for PFOA Detection

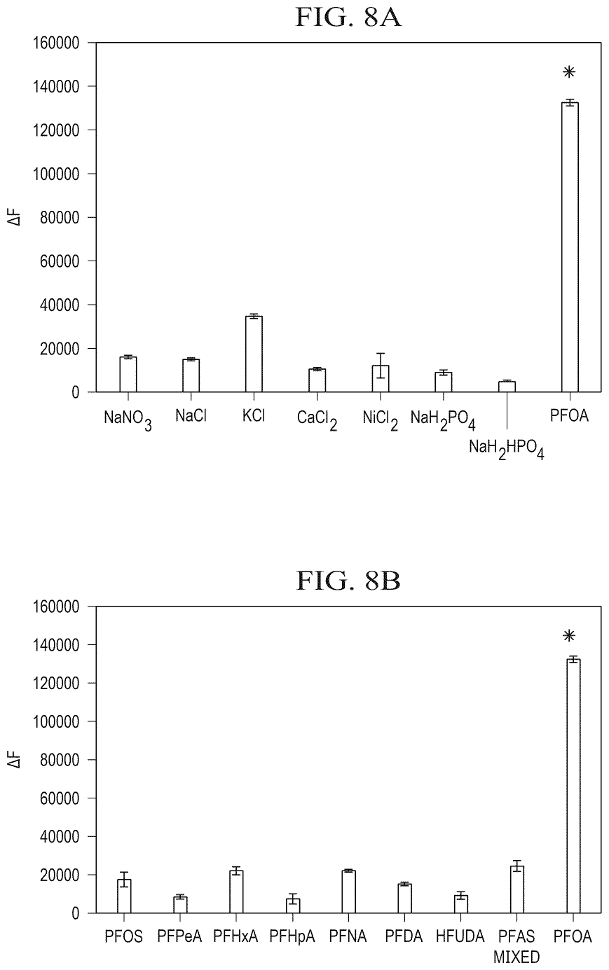

As shown in FIGS. 8A-8B, to assess the selectivity of the fluorescent SA hydrogel F-CDs, seven perfluorinated compounds, including PFOS, PFPeA, PFHxA, PFHpA, PFNA, PFDA, HFUDA, and a mixture of these compounds without PFOA-were evaluated under optimal conditions as substitutes for PFOA. As shown in FIG. 8A, PFOA induced a significant increase in fluorescence intensity, while the other compounds and their mixture exhibited minimal or negligible effects. To investigate the interfering effects of predominant water ionic compounds, FIG. 8B illustrates the fluorescence intensity enhancement (ΔF) of SA hydrogel F-CDs in the presence of various potential interfering substances. PFOA was detected at a concentration of 1.6 ppt, while inorganic chemicals were tested at 3.0 mg/mL. The minimal impact of these interfering substances on the fluorescence response highlights the excellent selectivity of the SA hydrogel F-CDs composite for PFOA detection.

### Sensitivity Assessment of the PFOA Detection Method

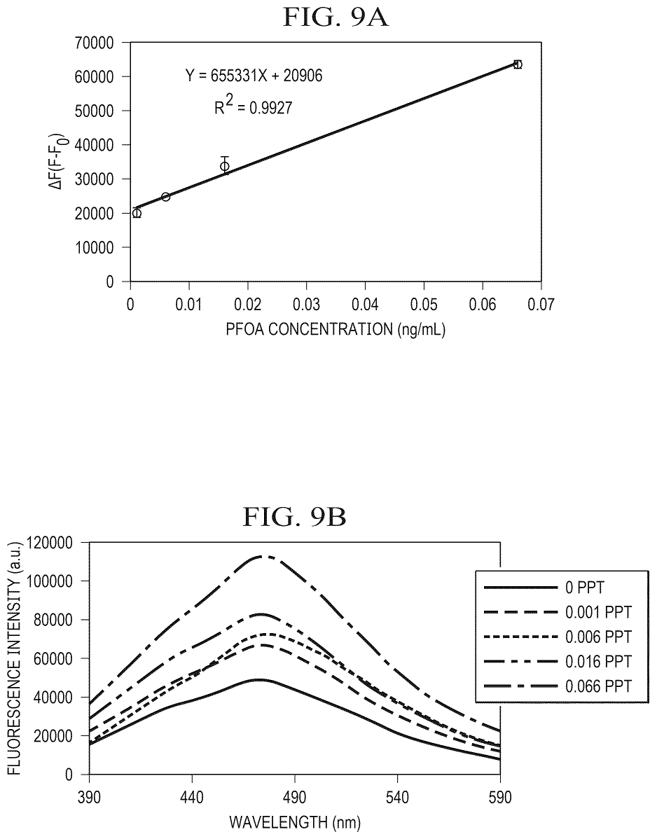

As shown in FIGS. 9A-9B, to determine the sensitivity of the detection method for PFOA, a series of concentrations ranging from 1 to 66 ppq were applied to the sensing system under optimal conditions. The calibration curve, as depicted in FIG. 9A, was constructed by plotting the variation in fluorescence intensity (ΔF=F−F0) against PFOA concentrations (cPFOA). The linear regression analysis yielded the equation ΔFX10⁻⁴=6.5*(cPFOA)+2, with a high correlation coefficient (R²=0.9927), indicating a linear response across the concentration range tested. Fluorescence emission spectra of SA hydrogel F-CDs at different PFOA concentrations are shown in FIG. 9B, demonstrating the increase in fluorescence intensity with increasing PFOA concentration. Table 2 presents a comparison of the detection performance of the SA hydrogel-F-CDs sensor with other previously reported methods for detecting PFOA and PFOS. The methods include techniques based on absorbance, colorimetry, interfacial tension measurement, electrochemical sensing, and fluorescence assays. Among these, electrochemical detection methods demonstrate particularly high sensitivity for the direct detection of PFOA. In contrast, the SA hydrogel-F-CDs developed in this study achieve a significantly lower limit of detection (LOD) compared to other methods, showcasing its enhanced sensitivity and effectiveness for PFOA detection.

TABLE 2
Comparison of different sensing methods
for PFAS detection, including absorbance,
colorimetry, electrochemical sensing, and fluorescence.

| Method | Nanomaterials | LOD | Linear range |
|---|---|---|---|
| Absorbance | Graphene | 0.4 ppb | 0-2 ppb |
| | oxide | | |
| Colorimetry | N-doped | — | 0.07-90 μM |
| | biomass carbon | | |
| | fiber | | |
| Colorimetry | Copper- | 14.3 nM | 50 nM-5 μM |
| | substituted | | |
| | cobalt-based | | |
| | Prussian blue | | |
| | analogue | | |
| | nanoboxes | | |
| | (CuCo-PBA | | |
| | NBs) | | |
| Interfacial | — | 4.5 μg L⁻¹ | 4.5 μg L⁻¹- |
| tensions | | | 600 mg L⁻¹ |
| Electrochemical | — | 1.08 μg L⁻¹ | 5-150 μg L⁻¹ |
| sensing | | | |
| Fluorescence | Eu/Tb- | 0.98 nM | 0.02-2.8 μM |
| | MOF@MIPs | | |
| Fluorescence | Cerium-oxo | 0.24 μg L⁻¹ | 0.5-7 μg L⁻¹ |
| | clusters | | |
| Fluorescence | PtCo | 7 μg L⁻¹ | 0.021-20.0 |
| | nanoparticle | | ppm |
| | (PtCoNPs)- | | |
| | decorated | | |
| | graphitic | | |
| | carbon nitride | | |
| | nanosheet | | |
| | (PtCoNPs@g- | | |
| | C₃N₄) | | |
| Electrochemical | Au NPs | 0.3 μM | 1 μM-100 |
| sensing | | | nM |
| Electrochemical | Ag NPs | 33 ppq | 50 ppq-1.6 |
| sensing | | | ppt |
| Fluorescence | SA hydrogel | | 0.001-0.066 |
| | F-CDs | | ppt |

Mechanistic Insights into Surface Charge Modulation and PFOA Detection

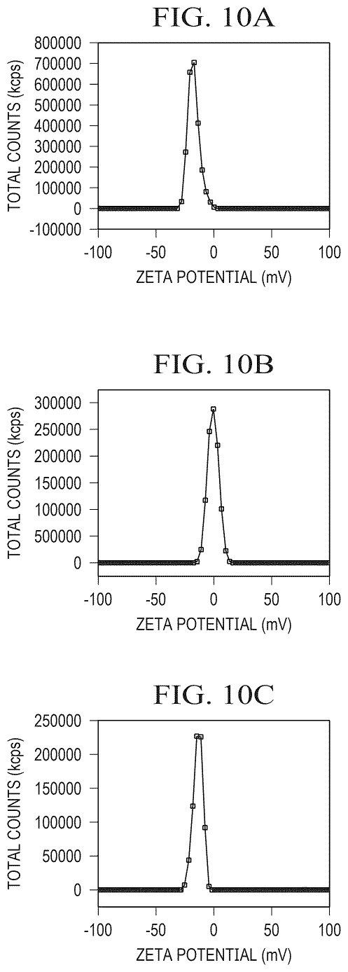

The surface charge properties of the SA hydrogel, SA hydrogel-F-CD, and SA hydrogel-F-CD in the presence of 0.1 ppb PFOA were assessed through zeta potential measurements, as shown in FIGS. 10A-10C. According to our recently published paper, the zeta potential of F-CDs alone was measured to be +18.57±2.6 mV, indicating a positive surface charge due to the functional groups present on the F-CDs. In contrast, the zeta potential of the bare SA hydrogel was −17.42±1.33 mV, reflecting its negatively charged surface, primarily attributed to the carboxyl groups in the alginate backbone, as shown in FIG. 10A. Upon incorporating F-CDs into the SA hydrogel, the zeta potential shifted significantly to −0.63±3.1 mV, as shown in FIG. 10B, indicating the neutralizing effect of the positively charged F-CDs on the negatively charged alginate matrix. This shift suggests strong interactions between the F-CDs and the hydrogel network. However, when 0.1 ppb PFOA was introduced to the SA hydrogel-F-CDs system, the zeta potential decreased again to −13.91±1.05 mV, as shown in FIG. 9C, implying that PFOA adsorption partially restored the negative surface charge. This decrease supports the hypothesis of strong electrostatic or fluorophilic interactions between PFOA and the F-CD-modified hydrogel, confirming the efficacy of the SA hydrogel-F-CDs composite in detecting PFOA through charge modulation.

### Fluorescence Sensing for PFOA in Potable Water

To demonstrate the practical applicability of the fluorescence sensing platform for PFOA detection, spiked recovery experiments were conducted using tap water collected from various locations in El Paso, Texas USA. The water samples were pre-treated by heating for 5 minutes and filtering through a 0.22 μm membrane. PFOA was then added to each sample to achieve final concentrations of 0, 1, 6, and 16 ppq for quantitative analysis. As shown in Table 3, the method yielded satisfactory recoveries ranging from 94.0% to 106.6%, with relative standard deviations (RSDs) between 0.4% and 5.2%, confirming the method's reliability for detecting PFOA in drinking water samples. Additionally, the amount of PFOA detected by the method was 1.751±0.02 ppq after diluting the water samples 1000-fold.

TABLE 3
Recovery rates of PFOA in spiked tap water samples
from different locations in El Paso.

| | Found | Spiked | | | |
|---|---|---|---|---|---|
| Samples | (ppq) | (ppq) | Detected (ppq) | Recovery % | RSD % |
| Tap | 1.78 | 1 | 2.76, 2.81, 2.72 | 98, 103, 94 | 1.6 |
| water | | 6 | 7.82, 7.5, 8.05 | 100.7, 95.3, 104.5 | 3.2 |
| 1 | | 16 | 17.58, 17.05, | 98.75, 95.4, 99.88 | 2.1 |
| | | | 17.76 | | |
| Tap | 1.744 | 1 | 2.75, 2.74, 2.76 | 100.6, 99.6, 101.6 | 0.3 |
| water | | 6 | 7.51, 8.14, 7.39 | 96.1, 106.6, 94.1 | 5.2 |
| 2 | | 16 | 17.99, 17.57, | 101.5, 98.9, 100.4 | 1.2 |
| | | | 17.81 | | |
| Tap | 1.733 | 1 | 2.777, 2.796, | 104.4, 106.3, 98.6 | 1.4 |
| water | | | 2.719 | | |
| 3 | | 6 | 7.536, 7.772, | 96.72, 100.65, | 1.9 |
| | | | 7.518 | 96.42 | |
| | | 16 | 16.985, 17.82. | 95.32, 100.54, | 2.6 |
| | | | 17.12 | 96.17 | |
| Tap | 1.747 | 1 | 2.798, 2.762, | 105.1, 101.5, 98.5 | 1.2 |
| water | | | 2.732 | | |
| 4 | | 6 | 7.717, 7.609, | 99.5, 97.7, 96.78 | 1.1 |
| | | | 7.554 | | |
| | | 16 | 17.15, 16.995, | 96.27, 95.05, | 0.7 |
| | | | 17.221 | 96.71 | |

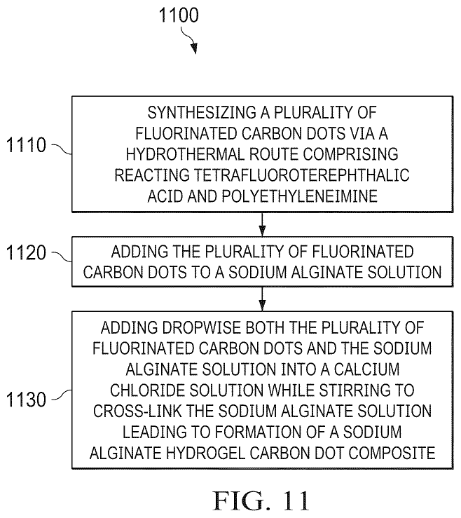

Turning to FIG. 11, method 1100 of making a sodium alginate hydrogel-carbon dot composite includes the following blocks. Block 1110 includes synthesizing a plurality of fluorinated carbon dots via a hydrothermal route comprising reacting tetrafluoroterephthalic acid and polyethyleneimine. Block 1120 includes adding the plurality of fluorinated carbon dots to a sodium alginate solution. Block 1130 includes, after adding the plurality of fluorinated carbon dots to a sodium alginate solution, adding dropwise both the plurality of fluorinated carbon dots and the sodium alginate solution into a calcium chloride solution while stirring to cross-link the sodium alginate solution leading to formation of a sodium alginate hydrogel-carbon dot composite. The calcium ions interact with a plurality of carboxylic groups of the sodium alginate solution by replacing at least some of sodium ions in the plurality of carboxylic groups of the sodium alginate solution with calcium ions creating a stable three-dimensional network. The fluorinated carbon dots are entrapped within an alginate network.

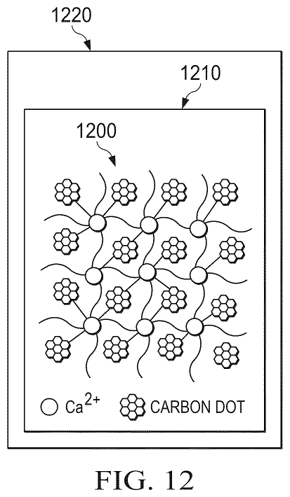

Turing to FIG. 12, an apparatus for detection of poly- and perfluoroalkyl substances in water is illustrated in schematic form. Device 1220 includes sensor 1210 which in-turn includes composition of matter 1200 comprising a sodium alginate hydrogel-fluorinated carbon dots composite comprising fluorinated carbon dots. The calcium ions replace at least some (all as illustrated) of sodium ions in carboxylic groups of the sodium alginate hydrogel-fluorinated carbon dots composite creating a stable three-dimensional network. The fluorinated carbon dots are entrapped within an alginate network.

The SA hydrogel-F-CDs composite provides a highly sensitive and selective platform for detecting trace amounts of PFOA and other PFAS in water samples. The incorporation of fluorinated carbon dots significantly enhances the fluorescence response, enabling detection at ultra-low concentrations, with a detection limit of 0.001 ppt. Characterization results confirm the successful integration of F-CDs within the hydrogel matrix, contributing to the stability and functionality of the sensor. The platform demonstrates excellent selectivity for PFOA among other perfluorinated compounds, showing minimal interference from potential contaminants. Practical testing with spiked tap water samples highlights the sensor's effectiveness in real-world applications, achieving high recovery rates and low relative standard deviations. This fluorescent hydrogel sensor represents a promising tool for environmental monitoring and the detection of persistent organic pollutants, offering advantages in sensitivity, specificity, and ease of use.

### Advantages

Embodiments of the present disclosure can be cost effective and advantageous for at least the following reasons. Embodiments offer several significant benefits over existing PFAS detection technologies.

For example, embodiments can provide ultra-high sensitivity. The device can detect PFAS, specifically perfluorooctanoic acid (PFOA), at ultra-low concentrations, down to 0.001 parts per trillion (ppt). This sensitivity surpasses many current technologies that may struggle to identify such trace levels, ensuring earlier detection and more accurate monitoring of potential water contamination.

As another example, embodiments can provide simple and cost-effective operation. The design of the fluorescence-based sensor makes it relatively easy and cost-effective to use compared to other advanced detection methods. It does not require extensive technical expertise, making it accessible for widespread application by water quality monitoring agencies and facilities.

As another example, embodiments can provide high selectivity. The sensor demonstrates high selectivity for PFOA, with minimal interference from other perfluorinated compounds or substances. This feature ensures that the device accurately identifies and quantifies PFOA without cross-reactivity issues common in other methods.

As another example, embodiments can provide compliance with Regulatory Standards. Embodiments can meet and exceed detection requirements established by the EPA for PFAS, making it an effective tool for regulatory compliance. Its precision in detecting low levels supports initiatives to adhere to the latest water quality guidelines and advisories.

Embodiments of the present disclosure improve quality and/or reduce costs compared to previous approaches.

### Practical Applications

The following are practical applications of embodiments of the present disclosure that have value within the technological arts of detection and monitoring for PFAS in water sources.

For example, embodiments can include a portable PFAS Detection Kit. This can be a compact, user-friendly testing kit for field use, enabling water quality inspectors, environmental agencies, and non-expert users to perform rapid on-site PFAS testing. This product would include the fluorescence sensor, disposable sample containers, and instructions for quick detection.

As another example, embodiments can include an integrated water monitoring system. This can be a continuous monitoring device that can be installed in water treatment plants, municipal water systems, or industrial facilities. This system would provide real-time data on PFAS levels, automatically alerting operators when concentrations exceed safe thresholds as set by regulatory bodies like the EPA.

As another example, embodiments can include a handheld PFAS detector. This can be a portable, battery-operated handheld device designed for easy transport and field analysis. This product would target environmental professionals and field researchers who need a reliable and sensitive tool for spot-checking various water bodies for PFAS contamination.

As another example, embodiments can include a consumer home water safety monitor. This can be a version of the detection device adapted for household use, allowing homeowners to check the safety of their tap water for PFAS levels. This product would appeal to consumers concerned with personal and family health.

As another example, embodiments can include a water filtration companion sensor. This can be an accessory that can be incorporated into water filtration systems to verify their effectiveness in removing PFAS in real-time. The sensor would indicate the presence of PFAS in the output water, providing additional assurance of filter performance.

As another example, embodiments can include a smart water quality analysis application. This can be a smartphone-integrated device that pairs with a mobile application for data visualization and analysis. Users could monitor water quality metrics, store historical data, and receive alerts and recommendations based on the PFAS levels detected.

As another example, embodiments can include educational lab kits. These can be kits designed for educational institutions to demonstrate the detection of PFAS in water using fluorescence-based sensors. These kits could be part of environmental science curriculums, allowing students to learn about water pollution and detection methods through hands-on experience.

As another example, embodiments can include an industrial wastewater monitor. This can be a sensor system for industries that discharge wastewater, ensuring compliance with environmental regulations. This system would track PFAS levels in discharge streams to prevent environmental contamination and meet government standards.

There are virtually innumerable uses for embodiments of the present disclosure, all of which need not be detailed here.

### Definitions

The term compound is intended to mean a substance formed when two or more chemical elements are chemically bonded together, the elements present in ratios with a limited range of variation and characteristic crystal structure.

The term uniformly is intended to mean unvarying or deviating very little from a given and/or expected value (e.g, within 10% of). The term substantially is intended to mean largely but not necessarily wholly that which is specified. The term approximately is intended to mean at least close to a given value (e.g., within 10% of). The term generally is intended to mean at least approaching a given state. The term coupled is intended to mean connected, although not necessarily directly, and not necessarily mechanically. The term proximate, as used herein, is intended to mean close, near adjacent and/or coincident; and includes spatial situations where specified functions and/or results (if any) can be carried out and/or achieved. The term distal, as used herein, is intended to mean far, away, spaced apart from and/or non-coincident, and includes spatial situation where specified functions and/or results (if any) can be carried out and/or achieved. The term deploying is intended to mean designing, building, shipping, installing and/or operating.

The terms first or one, and the phrases at least a first or at least one, are intended to mean the singular or the plural unless it is clear from the intrinsic text of this document that it is meant otherwise. The terms second or another, and the phrases at least a second or at least another, are intended to mean the singular or the plural unless it is clear from the intrinsic text of this document that it is meant otherwise. Unless expressly stated to the contrary in the intrinsic text of this document, the term or is intended to mean an inclusive or and not an exclusive or. Specifically, a condition A or B is satisfied by any one of the following: A is true (or present) and B is false (or not present), A is false (or not present) and B is true (or present), and both A and B are true (or present). The terms a and/or an are employed for grammatical style and merely for convenience.

The term plurality is intended to mean two or more than two. The term any is intended to mean all applicable members of a set or at least a subset of all applicable members of the set. The phrase any integer derivable therein is intended to mean an integer between the corresponding numbers recited in the specification. The phrase any range derivable therein is intended to mean any range within such corresponding numbers. The term means, when followed by the term “for” is intended to mean hardware, firmware and/or software for achieving a result. The term step, when followed by the term “for” is intended to mean a (sub) method, (sub) process and/or (sub) routine for achieving the recited result. Unless otherwise defined, all technical and scientific terms used herein have the same meaning as commonly understood by one of ordinary skill in the art to which this present disclosure belongs. In case of conflict, the present specification, including definitions, will control.

The described embodiments and examples are illustrative only and not intended to be limiting. Although embodiments of the present disclosure can be implemented separately, embodiments of the present disclosure may be integrated into the system(s) with which they are associated. All the embodiments of the present disclosure disclosed herein can be made and used without undue experimentation in light of the disclosure. Embodiments of the present disclosure are not limited by theoretical statements (if any) recited herein. The individual steps of embodiments of the present disclosure need not be performed in the disclosed manner, or combined in the disclosed sequences, but may be performed in any and all manner and/or combined in any and all sequences. The individual components of embodiments of the present disclosure need not be formed in the disclosed shapes, or combined in the disclosed configurations, but could be provided in any and all shapes, and/or combined in any and all configurations. The individual components need not be fabricated from the disclosed materials, but could be fabricated from any and all suitable materials. Homologous replacements may be substituted for the substances described herein. Agents which are chemically related may be substituted for the agents described herein where the same or similar results would be achieved.

Various substitutions, modifications, additions and/or rearrangements of the features of embodiments of the present disclosure may be made without deviating from the scope of the underlying inventive concept. All the disclosed elements and features of each disclosed embodiment can be combined with, or substituted for, the disclosed elements and features of every other disclosed embodiment except where such elements or features are mutually exclusive. The scope of the underlying inventive concept as defined by the appended claims and their equivalents cover all such substitutions, modifications, additions and/or rearrangements.

The appended claims are not to be interpreted as including means-plus-function limitations, unless such a limitation is explicitly recited in a given claim using the phrase(s) “means for” or “mechanism for” or “step for”. Sub-generic embodiments of this disclosure are delineated by the appended independent claims and their equivalents. Specific embodiments of this disclosure are differentiated by the appended dependent claims and their equivalents.

What is claimed is:

1. A method of making a sodium alginate hydrogel-carbon dot composite, comprising:
synthesizing a plurality of fluorinated carbon dots via a hydrothermal route comprising reacting tetrafluoroterephthalic acid and polyethyleneimine; and
adding the plurality of fluorinated carbon dots to a sodium alginate solution.

2. The method of claim 1, further comprising, after adding the plurality of fluorinated carbon dots to a sodium alginate solution, adding dropwise both the plurality of fluorinated carbon dots and the sodium alginate solution into a calcium chloride solution while stirring to cross-link the sodium alginate solution leading to formation of a sodium alginate hydrogel-carbon dots composite,
wherein calcium ions interact with a plurality of carboxylic groups of the sodium alginate solution by replacing at least some of sodium ions in the plurality of carboxylic groups of the sodium alginate solution with calcium ions creating a stable three-dimensional network, and
wherein the fluorinated carbon dots are entrapped within an alginate network.

3. The method of claim 2, wherein the calcium chloride solution has a concentration of approximately 0.1 mol/L CaCl₂.

4. The method of claim 1, wherein a ratio of tetrafluoroterephthalic acid to polyethyleneimine is approximately 3:1 by weight.

5. The method of claim 1, further comprising:
before adding the plurality of fluorinated carbon dots to a sodium alginate solution, stirring the sodium alginate solution and then filtering the sodium alginate solution; and
after adding the plurality of fluorinated carbon dots, stirring the plurality of fluorinated carbon dots in the sodium alginate solution.

6. The method of claim 5, wherein stirring comprises stirring a 2% sodium alginate solution at approximately 40° C. for approximately 4 hours.

7. The method of claim 5, wherein filtering comprises filtration through an approximately 0.4 um filter.

8. The method of claim 2, wherein the sodium alginate hydrogel-carbon dot composite comprises a luminescent sodium alginate hydrogel-carbon dot composite that is fluorescent in a presence of poly- and perfluoroalkyl substances in water.

9. An apparatus for detection of poly- and perfluoroalkyl substances in water, comprising
a device comprising a sensor comprising a sodium alginate hydrogel-fluorinated carbon dots composite comprising fluorinated carbon dots.

10. The apparatus of claim 9, wherein calcium ions replace at least some of sodium ions in carboxylic groups of the sodium alginate hydrogel-fluorinated carbon dots composite creating a stable three-dimensional network, and
wherein the fluorinated carbon dots are entrapped within an alginate network.

11. The apparatus of claim 9, wherein the sodium alginate hydrogel-fluorinated carbon dots composite exhibits optimal excitation at approximately 360 nm, with a corresponding emission peak at approximately 480 nm.

12. The apparatus of claim 11, wherein the sodium alginate hydrogel-fluorinated carbon dots composite presents statistically significant increased fluorescence in a presence of poly- and perfluoroalkyl substances in water compared to water.

13. The apparatus of claim 12, wherein the sodium alginate hydrogel-fluorinated carbon dots composite presents statistically significant increased fluorescence in the presence of poly- and perfluoroalkyl substances at a concentration of approximately 1.6 ppt in water compared to water.

14. The apparatus of claim 12, wherein the sodium alginate hydrogel-fluorinated carbon dots composite presents an approximately linear fluorescence response with respect to concentration of poly- and perfluoroalkyl substances in water.

15. A composition of matter, comprising:
a sodium alginate hydrogel-carbon dot composite comprising fluorinated carbon dots.

16. The composition of matter of claim 15, wherein calcium ions replace at least some sodium ions in carboxylic groups of the sodium alginate hydrogel-carbon dot composite creating a stable three-dimensional network, and
wherein the fluorinated carbon dots are entrapped within an alginate network.

17. The composition of matter of claim 15, wherein the sodium alginate hydrogel-fluorinated carbon dots composite exhibits optimal excitation at approximately 360 nm, with a corresponding emission peak at approximately 480 nm.

18. The composition of matter of claim 17, wherein the sodium alginate hydrogel-carbon dot composite presents statistically significant increased fluorescence in a presence of poly- and perfluoroalkyl substances in water compared to water.

19. The composition of matter of claim 18, wherein the sodium alginate hydrogel-carbon dot composite presents statistically significant increased fluorescence in the presence of poly- and perfluoroalkyl substances at a concentration of approximately 1.6 ppt in water compared to water.

20. The composition of matter of claim 18, wherein the sodium alginate hydrogel-carbon dot composite presents an approximately linear fluorescence response with respect to concentration of poly- and perfluoroalkyl substances in water.
